(19) United States
(12) Patent Application Publication
(10) Pub. No.: US 2015/0045400 A1
(43) Pub. Date: Feb. 12, 2015

(54) RITONAVIR COMPOSITIONS

(71) Applicants: Bandi PARTHASARADHI REDDY, Hyderabad, Andhrapradesh (IN); Podili KHADGAPATHI, Hyderabad, Andhra Pradesh (IN); Goli KAMALAKAR REDDY, Hyderabad, Andhra Pradesh (IN); HETERO RESEARCH FOUNDATION, Hyderabad, Andhra Pradesh (IN)

(72) Inventors: Bandi Parthasaradhi, Hyderabad (IN); Podili Khadgapathi, Hyderabad (IN); Goli Kamalakar Reddy, Andhra Pradesh (IN)

(21) Appl. No.: 14/382,075

(22) PCT Filed: Feb. 18, 2013
(86) PCT No.: PCT/IN13/00098

§ 371 Date: Aug. 29, 2014

(30) Foreign Application Priority Data
Mar. 1, 2012 (IN) 793/CHE/2012

(51) Int. Cl. A61K 31/427; A61K 47/32; A61K 45/06

(52) U.S. Cl. CPC A61K 31/427; A61K 45/06; A61K 47/32
USPC 514/365

(57) ABSTRACT

The present invention relates to pharmaceutical compositions comprising ritonavir premix, a water soluble polymer and a surfactant and process for preparing the same. More particularly, the present invention relates to hot-melt extrusion process for preparing solid oral compositions of ritonavir premix.

### PRIORITY

This patent application claims priority to Indian patent application number 793/CHE/2012, filed on Mar. 1, 2012, the contents of which are incorporated by reference herein in their entirety.

### FIELD OF THE INVENTION

Technical field of the present invention relates to pharmaceutical composition comprising ritonavir premix, a water soluble polymer and a surfactant; prepared by hot melt extrusion method.

### BACKGROUND

Chemically ritonavir is 10-Hydroxy-2-methyl-5-(1-methylethyl)-1-[2-(1-methylethyl) -4thiazolyl]-3,6-dioxo-8,11-bis((phenylmethyl)-2,4,7,12-tetraazatridecan-13-oic acid, 5-thiazolylmethyl ester, [5S-(5R*,8R*,10R*,11R*)]. Its empirical formula is: C₃₇H₄₈N₆O₅S₂, corresponding to a molecular weight of 720.95 and having the following structural formula:

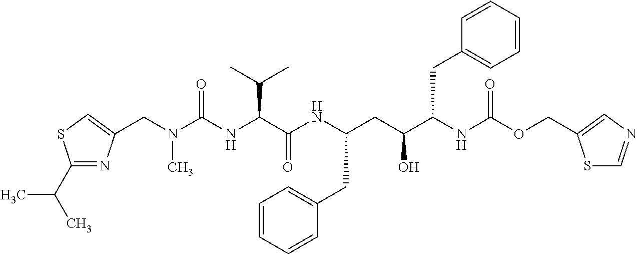 [SMILES: CC(C)c1nc(CN(C)C(=O)N[C@H](C(=O)N[C@@H](Cc2ccccc2)C[C@H](O)[C@H](Cc2ccccc2)NC(=O)OCc2cncs2)C(C)C)cs1]

Ritonavir is marketed under the trade name of NORVIR® in United States by Abbott in the form of 100 mg tablets, 100 mg capsules and 80 mg/ml oral solution for the treatment of human immunodeficiency virus (HIV).

Combination of Lopinavir and Ritonavir is marketed under the trade name of KALETRA® in United States by Abbott in the form of 200 mg:50 mg and 100 mg:25 mg tablets, 133.3 mg:33.3 mg capsules and 80 mg/ml:20 mg/ml oral solution.

U.S. Pat. No. 5,541,206 and U.S. Pat. No. 5,914,332 assigned to Abbott discloses ritonavir and lopinavir respectively.

U.S. Pat. No. 7,364,752 assigned to Abbott describes solvent evaporation method for preparing ritonavir compositions.

U.S. Pat. No. 8,025,899 assigned to Abbott claims melt-extrusion method for preparing a dosage form which includes solid dispersion comprising ritonavir, lopinavir, copovidone as water-soluble polymer and sorbitan monolaurate as surfactant.

U.S. Pat. No. 7,148,359 and U.S. Pat. No. 6,894,171 assigned to Abbott claims different polymorphs of ritonavir.

U.S. Pat. No. 7,205,413 assigned to TransForm pharmaceuticals describes crystalline Form III, IV and V of ritonavir.

An unpublished provisional application, IN 1803/CHE/2011 assigned to Hetero research foundation discloses amorphous ritonavir premix.

U.S. Pat. No. 5,635,523, U.S. Pat. No. 5,674,882, U.S. Pat. No. 5,886,036 and U.S. Pat. No. 6,284,767 assigned to Abbott describe combination of ritonavir and another HIV protease inhibiting compound for treating HIV infection.

U.S. Pat. No. 5,484,801, U.S. Pat. No. 5,948,436, U.S. Pat. No. 6,232,333, U.S. Pat. No. 7,141,593, U.S. Pat. No. 7,432,294, U.S. Pat. No. 6,458,818 and U.S. Pat. No. 6,521,651 assigned to Abbott describe pharmaceutical composition comprising solution of ritonavir.

U.S. Pat. No. 7,981,911 assigned to Abbott describes process for preparing ritonavir solution, to be filled into a capsule.

Still, there exists a need to develop new formulations of ritonavir with improved dissolution and bioavailability. Since, amorphous ritonavir have more permeability and hence more bioavailability compared to crystalline forms of ritonavir, inventors of the present invention have developed compositions of amorphous ritonavir premix with a water soluble polymer and a surfactant to improve dissolution and bioavailability which were also comparable with marketed NORVIR tablets.

### SUMMARY

One embodiment of the present invention provides pharmaceutical compositions comprising i) ritonavir premix, ii) a water-soluble polymer and iii) a surfactant wherein the composition is prepared by hot melt extrusion method.

In one aspect, ritonavir premix comprises ritonavir, copovidone, colloidal silicon dioxide and sorbitan monolaurate.

In another aspect, a process for preparing compositions of ritonavir premix by hot melt extrusion method involving: (i) sifting and blending ritonavir premix, water soluble polymer and one or more pharmaceutically acceptable excipients to form a dry mix, (ii) blending the dry mix of step no. (i) with surfactant, (iii) passing the material of step no. (ii) through hot melt extruder to form extrudes followed by milling and sifting and, (iv) blending the milled extrudes of step no. (iii) with remaining portion of excipients and finally compressing into tablets.

In yet another aspect of the present invention, water-soluble polymer is selected from copovidone and polyethylene oxide and said surfactant is selected from sorbitan monolaurate and polyoxyl 35 castor oil.

In one aspect, provides a solid oral composition in the form of a tablet comprising, based on the total weight of the composition, i) 10-25 wt % of ritonavir premix; ii) 30-65 wt % of the water soluble polymer selected from copovidone and polyethylene oxide; iii) 2-12 wt % of the surfactant selected from sorbitan monolaurate and polyoxyl 35 castor oil; and iv) optionally colloidal silicon dioxide, sodium stearyl fumarate, dibasic calcium phosphate; wherein the composition is prepared by hot melt extrusion method.

In another aspect, a solid oral composition comprising combination of ritonavir premix and at least one another HIV protease inhibitor selected from lopinavir, nelfinavir, saquinavir, atazanavir, amprenavir, fosamprenavir, tipranavir and darunavir; preferably lopinavir; wherein the composition is prepared by hot melt extrusion method.

In yet another aspect, the pharmaceutical composition comprising therapeutically effective amount of ritonavir is useful in treating HIV-infection.

### DETAILED DESCRIPTION

The present invention provides pharmaceutical compositions comprising ritonavir premix, a water-soluble polymer and a surfactant.

The term “effective amount” or “pharmaceutically effective amount” used interchangeably, is defined to mean the amount or quantity of the active drug (e.g. ritonavir), which is sufficient to elicit an appreciable biological response when administered to the patient. It will be appreciated that the precise therapeutic dose will depend on the age and condition of the patient, nature of the condition to be treated and will be at the ultimate discretion of the attendant physician.

As used in this specification and the appended claims, the singular forms “a”, “an”, and “the” include plural references unless the context clearly dictates otherwise. Thus for example, a reference to “a method” includes one or more methods, and/or steps of the type described herein and/or which will become apparent to those persons skilled in the art upon reading this disclosure and so forth.

The term “excipient” means a pharmacologically inactive component such as a diluent, disintegrant, carrier, etc of a pharmaceutical product. The excipients that are useful in preparing a pharmaceutical composition are generally safe, non-toxic and are acceptable for veterinary as well as human pharmaceutical use. Reference to an excipient includes both one and more than one such excipient.

The term “composition” or “pharmaceutical composition” or “solid oral composition” or “dosage form” as used herein synonymously include solid dosage forms such as tablets, capsules, granules, mini-tablets and the like meant for oral administration.

The present invention relates to pharmaceutical composition comprising i) ritonavir premix, ii) a water-soluble polymer and iii) a surfactant wherein the composition is prepared by hot melt extrusion method.

In accordance with the present invention, the term “ritonavir premix” comprises ritonavir, copovidone, colloidal silicon dioxide and sorbitan monolaurate, prepared as per the disclosure of an unpublished provisional application, IN 1803/CHE/2011 assigned to Hetero research foundation.

The present invention also provides process for preparing compositions of ritonavir premix by hot melt extrusion method involving: (i) sifting and blending ritonavir premix, water soluble polymer and one or more pharmaceutically acceptable excipients to form a dry mix, (ii) blending the dry mix of step no. (i) with surfactant, (iii) passing the material of step no. (ii) through hot melt extruder to form extrudes followed by milling and sifting and, (iv) blending the milled extrudes of step no. (iii) with remaining portion of excipients and finally compressing into tablets.

Water-soluble polymer according to the present invention is selected from copovidone and polyethylene oxide and said surfactant is selected from sorbitan monolaurate and polyoxyl 35 castor oil.

Solid oral composition according to the present invention is in the form of a tablet comprise, based on the total weight of the composition, i) 10-25 wt % of ritonavir premix; ii) 30-65 wt % of the water soluble polymer selected from copovidone and polyethylene oxide; iii) 2-12 wt % of the surfactant selected from sorbitan monolaurate and polyoxyl 35 castor oil; and iv) optionally colloidal silicon dioxide, sodium stearyl fumarate, dibasic calcium phosphate; wherein the composition is prepared by hot melt extrusion method.

The present invention relates to a solid oral composition comprising combination of ritonavir premix and at least one another HIV protease inhibitor selected from lopinavir, nelfinavir, saquinavir, atazanavir, amprenavir, fosamprenavir, tipranavir and darunavir; preferably lopinavir; wherein the composition is prepared by hot melt extrusion method.

Extrusion is defined as a process of converting raw material into a product of uniform shape and density by forcing it through a die under controlled conditions. The extrusion process can be operated in continuous manner and is capable of consistent product flow at relatively high throughput rates.

The melt-extrusion process comprises (i) sifting and blending an active ingredient, a water soluble polymer and one or more pharmaceutically acceptable excipients to form a dry mix, (ii) blending the dry mix of step no. (i) with surfactant, (iii) passing the material of step no. (ii) through hot melt extruder to form extrudes followed by milling and sifting and, (iv) blending the milled extrudes of step no. (iii) with remaining portion of excipients and finally compressing into tablets.

Suitable extruders include single screw extruder, twin screw extruder, intermeshing screw extruder, multiscrew extruder.

Suitable water-soluble polymer is selected from the group consisting of copolymer of N-vinyl pyrrolidone and vinyl acetate, polyethylene oxide, homopolymer of N-vinyl pyrrolidone, copolymer of N-vinyl pyrrolidone, copolymer of N-vinyl pyrrolidone and vinyl propionate, polyvinylpyrrolidone, methylcellulose, ethylcellulose, hydroxyalkylcelluloses, hydroxypropylcellulose, hydroxyalkylalkylcellulose, hydroxypropylmethylcellulose, cellulose phthalate, cellulose succinate, cellulose acetate phthalate, hydroxypropylmethylcellulose phthalate, hydroxypropylmethylcellulose succinate, hydroxypropylmethylcellulose acetate succinate, polypropylene oxide, copolymer of ethylene oxide and propylene oxide, methacrylic acid/ethyl acrylate copolymer, methacrylic acid/methyl methacrylate copolymer, butyl methacrylate/2-dimethylaminoethyl methacrylate copolymer, poly(hydroxyalkyl acrylate), poly(hydroxyalkyl methacrylate), copolymer of vinyl acetate and crotonic acid, partially hydrolyzed polyvinyl acetate, carrageenan, galactomannan, and xanthan gum.

Surfactants include for example, but are not limited to: sorbitan fatty acid mono esters such as sorbitan mono laurate (Span® 20), sorbitan monooleate, sorbitan monopalmitate (Span® 40), or sorbitan stearate; Polyoxyl 35 castor oil; polyoxyethylene alkyl ethers, e.g. polyoxyethylene lauryl ether, polyoxyethylene cetyl ether, polyoxyethylene stearyl ether; polyoxyethylene alkylaryl ethers, e.g. polyoxyethylene nonylphenyl ether, polyoxyethylene nonylphenyl ether, polyoxyethylene nonylphenyl ether, polyoxyethylene octylphenyl ether; polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (Lauroglycol®); or sucrose fatty acid esters, e.g. sucrose monostearate, sucrose distearate, sucrose monolaurate, sucrose dilaurate, or mixtures of one or more thereof.

Pharmaceutical compositions of ritonavir according to the present invention may further comprise one or more pharmaceutically acceptable excipients selected from diluent, disintegrant, glidant and lubricant.

Suitable diluents include dibasic calcium phosphate, tribasic calcium phosphate, calcium carbonate, calcium sulfate, magnesium carbonate, magnesium oxide, talc, lactose, sugar, starches, mannitol, sorbitol, inorganic salts, cellulose derivatives, calcium sulfate, xylitol, lactitol, kaolin, sucrose, mannitol, sorbitol, dextrates, dextrin, maltodextrin, dextrose and the like and mixtures thereof.

Suitable disintegrants include, by way of example and without limitation, colloidal silicon dioxide, croscarmellose sodium, crospovidone, sodium starch glycolate, carboxymethyl cellulose calcium, starches such as corn starch, potato starch, pre-gelatinized and modified starches, polacrillin potassium, polyvinylpyrrolidone, microcrystalline cellulose and the like or combinations thereof.

Suitable glidants include, by way of example and without limitation, colloidal silicon dioxide, calcium silicate, magnesium silicate, silicon hydrogel, cornstarch, talc and the like or combinations thereof.

Suitable lubricants include, by way of example and without limitation, sodium stearyl fumarate, calcium stearate, magnesium stearate, zinc stearate, stearic acid, fumaric acid, palmitic acid, talc, carnauba wax, hydrogenated vegetable oils, mineral oil, polyethylene glycols and the like or combinations thereof.

A film coat on the tablet provides an elegant appearance, protects from moisture and further contributes to the ease with which it can be swallowed.

The pharmaceutical composition comprising therapeutically effective amount of ritonavir as disclosed herein is useful for treating HIV-infection.

### EXAMPLES

The following examples further illustrate the invention and do not limit the scope of the invention.

### Example 1

### Ritonavir Table Compositions Prepared by Hot-Melt Extrusion Method

| | | |
|---|---|---|
| S. No | Ingredients | Mg/tablet |

Dry mix

| | | |
|---|---|---|
| 1. | Ritonavir premix^{#} | 133.33 |
| 2. | Copovidone | 425.52 |
| 3. | Colloidal silicon dioxide | 0.50 |

Addition of surfactant

| | | |
|---|---|---|
| 4. | Sorbitan monolaurate | 42.00 |

Pre-lubrication

| | | |
|---|---|---|
| 5. | Dibasic calcium phosphate | 74.75 |
| | anhydrous | |
| 6. | Colloidal silicon dioxide | 0.75 |

Lubrication

| | | |
|---|---|---|
| 7. | Sodium stearyl fumarate | 6.15 |

| | |
|---|---|
| Core tablet weight | 683.00 |

Film-coating

| | | |
|---|---|---|
| 8. | Opadry ® white | 17.07 |
| 9. | Purified water | q.s. |

| | |
|---|---|
| Coated tablet weight | 700.07 |

^{#}Each 133.33 mg of Ritonavir premix contains Ritonavir 100 mg.

### Manufacturing Process

i) Ritonavir premix, water soluble polymer and colloidal silicon dioxide were sifted through mesh #30,
ii) the sifted materials of step no. (i) were loaded into rapid mixer granulator and mixed for 10 minutes,
iii) surfactant was added to the materials of step no. (ii) while mixing for 6-7 minutes,
iv) the blend of step no. (iii) was passed through hot melt extruder to form extrudes,
v) the extrudes of step no. (iv) were milled using pulverizer and the milled extrudes were sifted through mesh #30,
vi) milled extrudes of step no. (v) were pre-lubricated with dibasic calcium phosphate anhydrous and colloidal silicon dioxide,
vii) pre-lubricated blend of step no. (vi) was lubricated with sodium stearyl fumarate and finally compressed into tablets and
viii) the tablets of step no. (vii) were film coated using Opadry® white.

### Dissolution Data for Example 1

Dissolution Medium: 60 mM polyoxyethylene-10-lauryl ether (POE10LE)
Volume: 900 ml
Apparatus: II (Paddle)
Speed: 75 RPM

| Time (min) | Cumulative % drug dissolved |
|---|---|
| 10 | 21 |
| 20 | 44 |
| 30 | 63 |
| 45 | 84 |
| 60 | 93 |
| 90 | 97 |
| 120 | 97 |
| 150 | 97 |
| 180 | 98 |

### Example 2

### Ritonavir Tablet Compositions Prepared by Hot-Melt Extrusion Method

| | | |
|---|---|---|
| S. No | Ingredients | Mg/tablet |

Dry mix

| | | |
|---|---|---|
| 1. | Ritonavir premix^{#} | 133.330 |
| 2. | Polyethylene oxide (Polyox) | 425.520 |
| 3. | Colloidal silicon dioxide | 0.500 |

Addition of surfactant

| | | |
|---|---|---|
| 4. | Sorbitan monolaurate | 42.000 |

Pre-lubrication

| | | |
|---|---|---|
| 5. | Dibasic calcium phosphate | 74.750 |
| | anhydrous | |
| 6. | Colloidal silicon dioxide | 0.750 |

Lubrication

| | | |
|---|---|---|
| 7. | Sodium stearyl fumarate | 6.150 |

| | |
|---|---|
| Core tablet weight | 683.00 |

Film-coating

| | | |
|---|---|---|
| 8. | Opadry white | 17.07 |
| 9. | Purified water | q.s. |

| | |
|---|---|
| Coated tablet weight | 700.07 |

^{#}Each 133.33 mg of Ritonavir premix contains Ritonavir 100 mg.

### Manufacturing Process

Same as given for Example 1.

### Example 3

### Ritonavir Table Compositions Prepared by Hot-Melt Extrusion Method

| | | |
|---|---|---|
| S. No | Ingredients | Mg/tablet |

Dry mix

| | | |
|---|---|---|
| 1. | Ritonavir premix^{#} | 133.330 |
| 2. | Copovidone | 425.520 |
| 3. | Colloidal silicon dioxide | 0.500 |

Addition of surfactant

| | | |
|---|---|---|
| 4. | Polyoxyl 35 castor oil | 42.000 |

Pre-lubrication

| | | |
|---|---|---|
| 5. | Dibasic calcium phosphate | 74.750 |
| | anhydrous | |
| 6. | Colloidal silicon dioxide | 0.750 |

Lubrication

| | | |
|---|---|---|
| 7. | Sodium stearyl fumarate | 6.150 |

| | |
|---|---|
| Core tablet weight | 683.00 |

Film-coating

| | | |
|---|---|---|
| 8. | Opadry white | 17.075 |
| 9. | Purified water | q.s. |

| | |
|---|---|
| Coated tablet weight | 700.07 |

^{#}Each 133.33 mg of Ritonavir premix contains Ritonavir 100 mg.

### Manufacturing Process

Same as given for Example 1.

### Example 4

### Tablet Composition Comprising Ritonavir and Lopinavir Prepared by Hot-Melt Extrusion Method

| | | |
|---|---|---|
| S. No | Ingredients | Mg/tablet |

Dry mix

| | | |
|---|---|---|
| 1. | Ritonavir premix^{#} | 66.66 |
| 2. | Lopinavir (amorphous) | 200.00 |
| 3. | Copovidone | 850.20 |
| 4. | Colloidal silicon dioxide | 12.00 |

Addition of surfactant

| | | |
|---|---|---|
| 5. | Sorbitan monolaurate | 80.90 |

Lubrication

| | | |
|---|---|---|
| 6. | Colloidal silicon dioxide | 18.00 |
| 7. | Sodium stearyl fumarate | 12.30 |

| | |
|---|---|
| Core tablet weight | 1240.00 |

Film-coating

| | | |
|---|---|---|
| 8. | Opadry ® yellow | 31.00 |
| 9. | Purified water | q.s. |

| | |
|---|---|
| Coated tablet weight | 1271.00 |

^{#}Each 66.66 mg of Ritonavir premix contains Ritonavir 50 mg.

### Manufacturing Process

i) Ritonavir premix, lopinavir, copovidone and colloidal silicon dioxide were sifted through mesh #30,
ii) the sifted materials of step no. (i) were loaded into rapid mixer granulator and mixed for 10 minutes,
iii) surfactant was added to the materials of step no. (ii) while mixing for 6-7 minutes,
iv) the blend of step no. (iii) was passed through hot melt extruder to form extrudes,
v) the extrudes of step no. (iv) were milled using pulverizer and the milled extrudes were sifted through mesh #30,
vi) milled extrudes of step no. (v) were lubricated with colloidal silicon dioxide and sodium stearyl fumarate and finally compressed into tablets and
vii) the tablets of step no. (vi) were film coated using Opadry® yellow.

### Example 5

### Tablet Composition Comprising Ritonavir and Lopinavir Prepared by Hot-Melt Extrusion Method

| | | |
|---|---|---|
| S. No | Ingredients | Mg/tablet |

Dry mix

| | | |
|---|---|---|
| 1. | Ritonavir premix^{#} | 66.66 |
| 2. | Lopinavir (amorphous) | 200.00 |
| 3. | Copovidone | 850.20 |
| 4. | Colloidal silicon dioxide | 12.00 |

Addition of surfactant

| | | |
|---|---|---|
| 5. | Polyoxyl 35 castor oil | 80.90 |

Lubrication

| | | |
|---|---|---|
| 6. | Colloidal silicon dioxide | 18.00 |
| 7. | Sodium stearyl fumarate | 12.30 |

| | |
|---|---|
| Core tablet weight | 1240.00 |

Film-coating

| | | |
|---|---|---|
| 8. | Opadry ® yellow | 31.00 |
| 9. | Purified water | q.s. |

| | |
|---|---|
| Coated tablet weight | 1271.00 |

^{#}Each 66.66 mg of Ritonavir premix contains Ritonavir 50 mg.

### Manufacturing Process

Same as given for example 4.

1. A pharmaceutical composition comprising i) ritonavir premix, ii) a water-soluble polymer and iii) a surfactant wherein the composition is prepared by hot melt extrusion method.

2. The pharmaceutical composition according to claim 1, wherein said ritonavir premix comprises ritonavir, copovidone, colloidal silicon dioxide and sorbitan monolaurate.

3. The pharmaceutical composition according to claim 1, further comprise a diluent, a disintegrant, a glidant, a lubricant, or a combination thereof.

4. The pharmaceutical composition according to claim 3, wherein diluent is selected from the group consisting of dibasic calcium phosphate, lactose, calcium carbonate, micro crystalline cellulose and combination thereof; disintegrant selected from colloidal silicon dioxide, croscarmellose sodium, crospovidone, sodium starch glycolate, carboxymethyl cellulose calcium and the like or combinations thereof; glidant selected from colloidal silicon dioxide, calcium silicate, magnesium silicate, silicon hydrogel, cornstarch, talc and the like or combinations thereof; lubricant selected from magnesium stearate, calcium stearate, zinc stearate, talc or mixtures thereof.

5. The pharmaceutical composition according to claim 1, selected from a tablet, a capsule and a granule.

6. A process for preparing compositions of ritonavir premix dosage form by hot melt extrusion method involves:
(i) sifting and blending ritonavir premix, water soluble polymer and one or more pharmaceutically acceptable excipients to form a dry mix,
(ii) blending the dry mix of step no. (i) with surfactant,
(iii) passing the material of step no. (ii) through hot melt extruder to form extrudes followed by milling and sifting and,
(iv) blending the milled extrudes of step no. (iii) with remaining portion of excipients and finally compressing into tablets.

7. The pharmaceutical composition according to claim 1, wherein said water-soluble polymer is selected from copovidone and polyethylene oxide and said surfactant is selected from sorbitan monolaurate and polyoxyl 35 castor oil.

8. A solid oral composition in the form of a tablet comprising, based on the total weight of the composition,
i) 10 to 25 wt% of ritonavir premix;
ii) 30 to 65 wt% of the water soluble polymer selected from copovidone and polyethylene oxide;
iii) 2 to 12 wt% of the surfactant selected from sorbitan monolaurate and polyoxyl 35 castor oil; and
iv) optionally colloidal silicon dioxide, sodium stearyl fumarate, dibasic calcium phosphate; wherein the composition is prepared by hot melt extrusion method.

9. A solid oral composition comprising combination of ritonavir premix and at least one another HIV protease inhibitor selected from lopinavir, nelfinavir, saquinavir, atazanavir, amprenavir, fosamprenavir, tipranavir and darunavir; preferably lopinavir; wherein the composition is prepared by hot melt extrusion method.

10. The pharmaceutical composition comprising therapeutically effective amount of ritonavir according to claim 1 useful in treating HIV-infection.
